(19) United States
(12) Patent Application Publication
(10) Pub. No.: US 2011/0051892 A1
(43) Pub. Date: Mar. 3, 2011

(54) RADIATION THERAPY SKIN MARKER

(75) Inventor: Helen Zinreich Shafer, Owings Mills, MD (US)

(73) Assignee: IZI MEDICAL PRODUCTS, LLC, Baltimore, MD (US)

(21) Appl. No.: 12/551,952

(22) Filed: Sep. 1, 2009

(51) Int. Cl. A61N 5/10; A61B 6/08

(52) U.S. Cl. 378/65; 378/205

(57) ABSTRACT

Radiation therapy skin markers used to delineate radiation therapy portal areas of a patient to be treated with radiation are described. The radiation therapy skin marker includes comprises a substrate having a first surface and a second surface. The first surface of the substrate is lined with an ink to facilitate the outlining of the treatment field. The second surface is coated with an adhesive layer which is protected by release liner.

The invention relates to devices used to delineate radiation therapy portal areas of patients to be treated with radiation. More particularly, the invention relates to markers used to delineate radiation therapy portal areas of patients to be treated with radiation. Even more particularly, the invention relates to a lined markers used to delineate radiation therapy portal areas of patients to be treated with radiation.

### BACKGROUND OF THE INVENTION

Radiation therapy is a common method in the treatment of those individuals diagnosed with cancer. Radiation therapy is used to treat cancer patients in two ways: for curative purposes and for palliative reasons.

Virtually all radiation therapy centers are equipped with simulators, which includes a fluoroscopic imaging unit equipped with all the characteristics and parameters found on the radiation treatment units. With the help of diagnostic imaging such as computerized x-ray tomography (CT) and magnetic resonance imaging (MRI), when combined with the fluoroscopic capability of the simulator, a radiation therapy portal, which is the area through that the treating radiation will be focused, may be designed.

Markings, also sometimes referred to as tattoos, are typically applied to the skin of a patient in preparation for radiation therapy or other medical treatment, as a means of clearly delineating the location of treatment set-up points. Traditionally, the marking is done by applying a drop of marking agent to the surface of the patient's skin and then penetrating the skin with a needle to allow the marking agent to flow under the surface of the skin. The size of the needle used can vary widely, some technicians preferring to work with a larger needle, others with a smaller needle. When marking the skin in this way, it is critical that the marking agent, such as India ink, as well as the needle tip be sterile. Furthermore, it is highly desirable to have a disposable needle, to eliminate the possibility of passing pathogens from one person to another and to eliminate the need for sterilizing the needles. It is also desirable to have a marking device that will accept a standard needle that is typically kept in stock in a healthcare facility, in a range of needle gauges.

The time between simulation and beginning treatment is usually zero to seven days. During this period, generally greater than one day, various types of markings may be lost. For this reason, tattooing is often used to establish the portal boundaries with some permanency and reproducibility. However, there are several disadvantages to tattooing, even though it may seem to be the most optimal means to establish portal boundaries.

One problem is that tattoos are difficult to recognize on dark skin. Thus, tattoos too may not be completely visible during the radiation therapy treatment.

An additional problem is that the tattooing process punctures the patient's skin multiple times and, therefore, has the potential to expose radiation therapy personnel to the patient's blood and the patient's blood to out-side contaminants. This exposure creates a risk that the radiation therapy personnel or the patient may be contaminated with various infectious organisms transmitted by blood exposure.

Two further problems are associated with the permanency of tattoos. First, a radiation therapy portal boundary tattoo is aesthetically unattractive. This is especially problematic when the tattoo is placed on skin surfaces which are not ordinarily covered by clothing. Second, but technically of equal importance, a radiation therapy portal boundary tattoo is inflexible. Initially, this may seem to be a benefit because it limits the risk that the portal boundary markings might inadvertently shift. However, a portal field for a particular patient is frequently changed throughout the course of treatment thereby requiring a shift in the portal boundary markings. Once tattooing marks are established a re-tattooing is required to shift the portal boundaries thereby compounding the above described disadvantages.

Notwithstanding the state of the art as described herein, there is a need for further improvements in transparent radiation therapy skin markers, which facilitate the precise application of the markers for radiation therapy treatment.

### SUMMARY OF THE INVENTION

In order to overcome shortcomings of techniques used to depict portal fields, a specially designed device is presented. This device is easy to apply to the skin of a patient. The device is reliable in that it may retain its original position from about seven and to about ten days. Yet, it may be repositioned easily without risk to patient or personnel. Furthermore, it is removable without leaving any permanent traces on the patient's skin.

The device comprises flat, adhesive-coated marker structures in various shapes which are used to denote the perimeter, isocenter(s), and set-up points of radiation treatment portals on the skin of patients undergoing radiation therapy. In one embodiment, the device includes pieces shaped such that they may be used to delineate the corners, edges connecting the corners, the isocenter point(s), and any set-up points of the proposed radiation field.

In general, one aspect of the invention is to provide a device for marking skin surfaces to delineate a radiation therapy portal area. The device includes a substrate comprising a first and second surface, wherein the first surface is lined with an ink to facilitate outlining of a radiation therapy treatment field, an adhesive layer coated onto the second surface of the substrate, and a removable release liner in contact with the adhesive layer.

Another aspect of the invention is to provide a method of marking a skin surface to delineate a radiation therapy portal area. The method includes the steps of providing at least one skin marker, removing a portion of a backing liner of a skin marker to expose an adhesive surface of the skin marker, positioning the at least one skin marker on a skin surface of a patient, wherein the positioning of the at least one skin marker delineates a radiation therapy portal area, and releasably securing the at least one skin marker in place on the skin surface of the patient.

### BRIEF DESCRIPTION OF THE DRAWINGS

FIG. 1 illustrates a cross-sectional view of a radiation therapy skin marker according to an embodiment of the invention;

FIG. 2 illustrates a bottom view of a radiation therapy skin marker according to an embodiment of the invention;

FIG. 3 illustrates a perspective view of an arrangement of radiation therapy skin markers for radiation treatment according to an embodiment of the invention;

FIG. 4 illustrates a line marker according to an embodiment of the invention;

FIG. 5 illustrates one embodiment of an isocenter marker according to an embodiment of the invention; and

FIG. 6 illustrates another embodiment of an isocenter marker according to an embodiment of the invention.

### DETAILED DESCRIPTION OF THE INVENTION

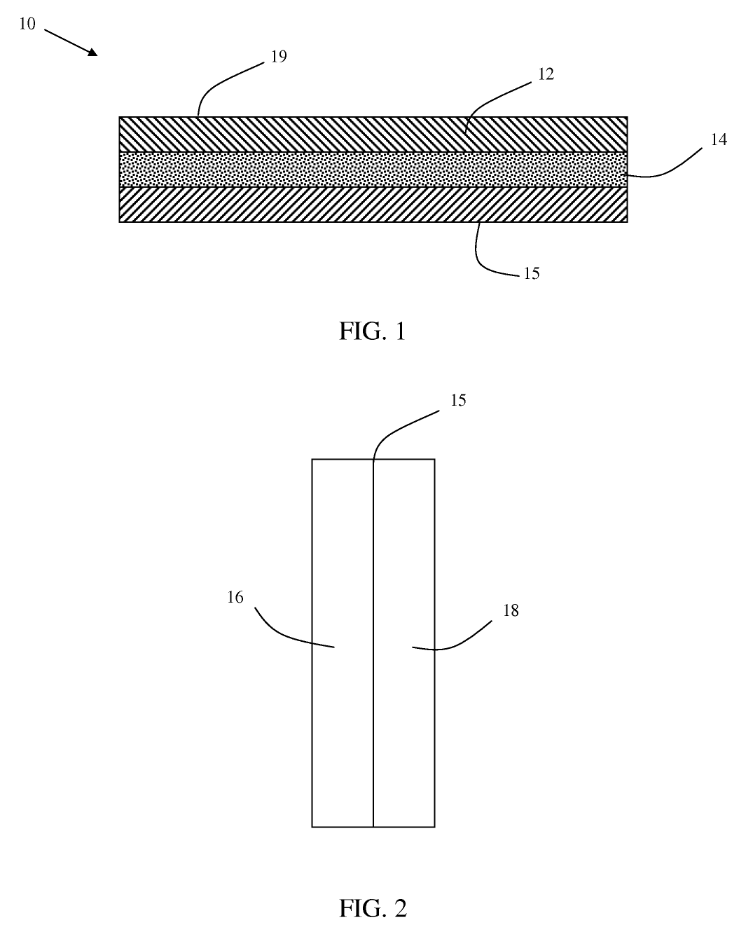

An embodiment of the invention relates to radiation therapy skin markers to delineate radiation therapy portal areas of a patient to be treated with radiation. As seen in FIGS. 1 and 2, a radiation therapy skin marker 10 comprises a substrate 12 having a first surface 19 and a second surface (not shown). Surface 19 of substrate 12 is lined with an ink to facilitate the outlining of the treatment field. The second surface is coated with an adhesive layer 14 which is protected by release liner 15, optionally divided into multiple segments, shown here as 16 and 18.

Substrate 12 of radiation therapy skin marker 10 is prepared from material that is both conformable to the contours of the body, and flexible so as to permit free movement of the body part wearing the product. Further, substrate 12 is lightweight, and is elastic in character. In an embodiment of the invention, substrate 12 is a non-woven substrate, such as a film having a thickness in the range from about 1 mil to about 3 mils. Substrate 12 may be prepared into films from materials including polyolefins, such as polyethylene, polyurethanes, and polyvinylchloride.

Adhesive layer 14 of radiation therapy skin marker 10 may be a medical grade adhesive. In one embodiment of the invention, the medical grade adhesive is a pressure sensitive adhesive. An “adhesive” as used herein means any natural or synthetic substance that is capable of surface attachment. As used herein, the term “pressure-sensitive adhesive” refers to a viscoelastic material which adheres instantaneously to most substrates with the application of very slight pressure and remains permanently tacky. Other conventional components may be added to the pressure sensitive adhesive including, but not limited to, inhibitors, fillers, pigments, stabilizers, tackifiers, UV absorbers, and the like.

The pressure sensitive adhesive of adhesive layer 14 may include mixtures of different polymers and mixtures of polymers, such as polyisobutylenes (PIB) of different molecular weights. The pressure sensitive adhesive may also be an acrylate or methacrylate based pressure sensitive adhesive. The adhesive is prepared according to standard industry procedures.

When the pressure sensitive adhesive is an acrylate or methacrylate based pressure sensitive adhesive, adhesive may be formed by a polymerization reaction product of at least two alkyl acrylate or methacrylate ester monomers, at least one ethylenically unsaturated carboxylic acid, at least one vinyl lactam, and most preferably including a crosslinking agent. Examples of suitable alkyl acrylate or methacrylate esters include, but are not limited to, butyl acrylate, ethyl acrylate, 2-ethylhexyl acrylate, isooctyl acrylate, isononyl acrylate, isodecyl acrylate, methyl acrylate, methylbutyl acrylate, 4-methyl-2-pentyl acrylate, sec-butyl acrylate, ethyl methacrylate, isodecyl methacrylate, methyl methacrylate, and the like, and mixtures thereof.

Examples of suitable ethylenically unsaturated carboxylic acids include, but are not limited to, acrylic acid, methacrylic acid, fumaric acid, itaconic acid, and the like, and mixtures thereof. In one embodiment, the ethylenically unsaturated carboxylic acid monomer is acrylic acid.

In one embodiment of the invention, the ink used for the line on surface 19 of substrate 12 includes a phosphorescent ink, a fluorescent ink, a dye-based ink, a pigment-based ink, and combinations thereof. In one embodiment, the color of the ink may be blue, red, green, white, black or combinations thereof. The ink may be applied to surface 19 of substrate 12 by a variety of printing and imaging methods including, but not limited to, flexography, lithography, rotogravure, screen printing, digital printing process or combinations thereof. The ink line on surface 19 of substrate 12 is highly visible, which facilitates the application of the radiation therapy skin marker 10 on a desired body surface location of a patient. The colored ink line is also useful for application of the radiation therapy skin marker 10 on a variety of patient skin colors.

Radiation therapy skin marker 10 is a skin-contact marker for which its conformability to the contours of the body and flexibility with body movement characteristics are most advantageous. The marker 10 is compatible with human skin, even during extended contact, and is both flexible and conformable. Adhesive layer 14 of radiation therapy skin marker 10 may exhibit sufficient adhesion, or bonding strength, to prevent peeling of the adhesive product from the skin prematurely, but is not tenacious as to damage or irritate the skin during removal, especially sensitive skin, or to leave adhesive residue on the skin. In one embodiment, marker 10 is designed to remain in a designated position on the skin of a patient for at least 7 to about 10 days. Suitable materials that may be used in the preparation of radiation therapy skin marker 10 include DM-8010 and DM-8014 which are manufactured by DermaMed Coatings Company, LLC.

In another embodiment of the invention, the radiation therapy skin marker, including substrate 12 and adhesive layer 14, is clear and transparent. In yet another embodiment of the invention, the radiation therapy skin marker, including substrate 12, adhesive layer 14, and release liner 15, is clear and transparent. In still yet another embodiment of the invention, at least a portion of the radiation therapy skin marker 10 is clear and transparent, thus forming a “zone of transparency”. The transparency of marker 10 facilitates precise application of marker 10 for radiation therapy treatment.

In one embodiment, radiation therapy skin marker 10 may be made by coating a layer of the pressure sensitive adhesive onto a liner, and then transferring (laminating) the adhesive onto substrate 12. The liner, such as a kraft paper, may be coated on one side with a release layer, such as a silicone compound, and is then coated in a first stage, with the adhesive on top of the release layer. The adhesive can be applied to the liner as a solution or emulsion of the adhesive formulation, from which the solvent or water is removed by heating or as a hot melt adhesive such as by casting or extrusion.

The substrate 12 may then be laminated to the adhesive layer, in a second stage. If substrate 12 is formed on a carrier, the substrate/carrier laminate is contacted with the adhesive/liner laminate, such that the substrate is interposed between its carrier on one side, and the adhesive layer 14 of the adhesive coated liner on the other side. The carrier is then stripped off of the laminate, and is discarded, thereby leaving a laminate of substrate 12, adhesive 14 and release liner.

Next, the liner is stripped off the laminate. The adhesive sticks to substrate 12, but not the liner. Simultaneously, a lightweight paper liner coated with a silicone material, which is slit to form release liner segments 16 and 18, is contacted with the laminate, and then the final laminate is passed to a cutter, or a die, to cut marker 10 to a desired length.

In one embodiment of the invention, the radiation therapy skin marker 10 may be provided in either roll form, strips or in sets of pre-cut lengths. A suitable dispenser for a roll of skin markers 10 may be used to dispense a desired length of skin marker 10. Such dispensers may separate release liner segments 16 and 18 from skin marker 10 prior to use. Packaging for a strip form or the set of pre-cut lengths of skin marker 10 may be provided in sterile, hermetically sealed packages for individual use.

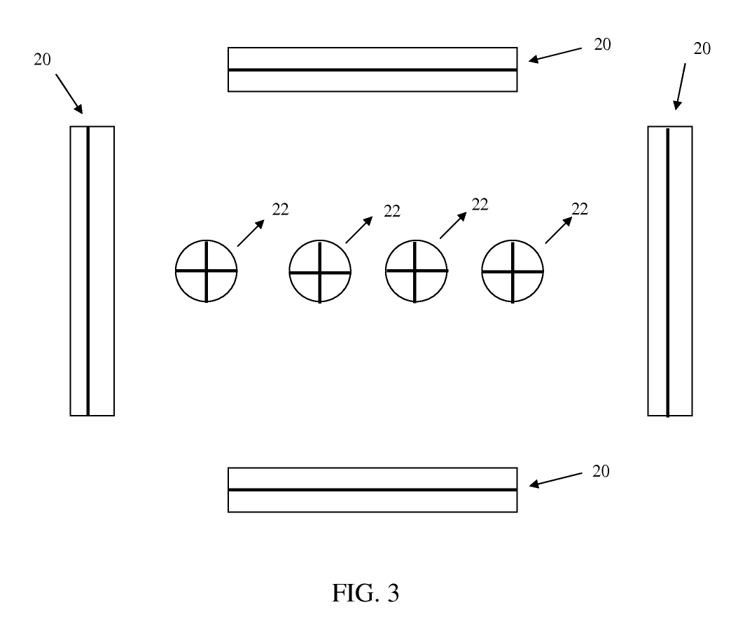

Examples of suitable radiation therapy skin markers 10 include a line marker 20 and an isocenter marker 22. The line markers 20 can be used, respectively, to mark the corners and edges of a square or rectangle radiation therapy portal, as shown in FIG. 3. The isocenter markers 22 can be used, respectively, to mark any isocenter and set-up points necessary to a particular radiation therapy treatment, as shown in FIG. 3.

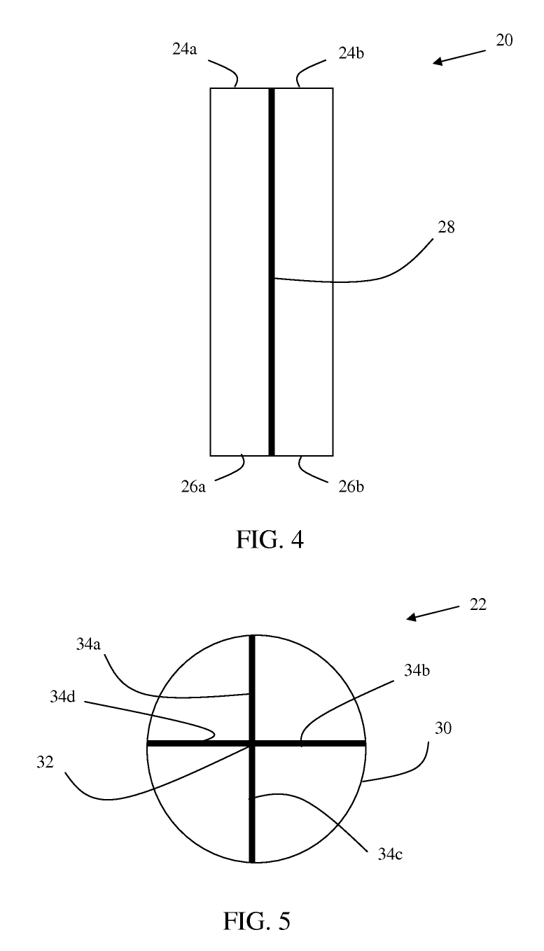

In one embodiment of the invention, as shown in FIG. 4, line marker 20 is somewhat rectangular in shape and defined by short edges 24a and 24b and long edges 26a and 26b. The long edges 26a and 26b are multiple times the length of the short edges 24a and 24b. Each line marker 20 includes a printed line 28 extending between the short edges 24a and 24b and parallel to the long edges 26a and 26b. The line markers 20 may placed by a radiation therapist such that the printed lines 28 of each line marker 20 outline the edges of a specified square or rectangular radiation therapy portal (as shown in FIG. 5). The printed lines 28 assist in creating a discernable target for a radiation therapist performing radiation therapy treatments.

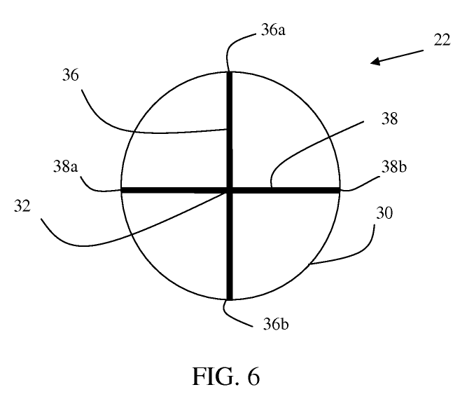

In another embodiment of the invention, as shown in FIG. 5, isocenter marker 22 is circularly shaped and is defined by a circular outer edge 30 and a midpoint 32. Each isocenter marker 22 includes four printed lines 34a-34d extending radially from the midpoint 32 to the circular outer edge 30. The printed lines 34a-34d are further defined in that they intersect at midpoint 32 in such a way that lines 34a and 34c would each be perpendicular to lines 34b and 34d. In an alternative embodiment, as seen in FIG. 6, each isocenter marker 22 includes at least two printed lines 36 and 38 that intersect to form midpoint 32. Line 36 extends from one point (36a) on circular outer edge 30 to another point (36b) on the circular edge 30. Line 38 extends from one point (38a) on circular outer edge 30 to another point (38b) on the circular edge 30. The printed lines 36 and 38 are further defined in that they intersect at midpoint 32 in such a way that line 36 is perpendicular to line 38. The isocenter markers 22 may be placed by a radiation therapist such that the printed lines 34a-34d, as well as lines 36 and 38, define an isocenter of a specific radiation therapy portal as the point where the lines 34a-34d, as well as lines 36 and 38, would intersect thereby assisting a radiation therapist perform radiation therapy treatments, as seen in FIG. 3.

Based upon the foregoing disclosure, it should now be apparent that the radiation therapy skin markers as described herein will carry out the objects set forth hereinabove. It is, therefore, to be understood that any variations evident fall within the scope of the claimed invention and thus, the selection of specific component elements can be determined without departing from the spirit of the invention herein disclosed and described.

What is claimed is:

1. A device for marking skin surfaces to delineate a radiation therapy portal area, the device comprising:
a substrate comprising a first and second surface, wherein the first surface is lined with an ink to facilitate outlining of a radiation therapy treatment field;
an adhesive layer coated onto the second surface of the substrate; and
a removable release liner in contact with the adhesive layer.

2. The device of claim 1, wherein the substrate and adhesive layer are transparent.

3. The device of claim 1, wherein at least a portion of the substrate and adhesive layer are transparent.

4. The device of claim 1, wherein the substrate comprises a material selected from the group consisting of a polyolefin, a polyurethane, and a polyvinylchloride.

5. The device of claim 4, wherein the substrate comprises a polyolefin.

6. The device of claim 5, wherein the polyolefin is polyethylene.

7. The device of claim 1, wherein the adhesive layer comprises a medical grade adhesive.

8. The device of claim 7, wherein the medical grade adhesive is a pressure sensitive adhesive.

9. The device of claim 1, wherein the device is a line marker having a rectangular shape.

10. The device of claim 1, wherein the device is an isocenter marker.

11. The device of claim 1, wherein the ink is selected from a group consisting of a phosphorescent ink, a fluorescent ink, a dye-based ink, a pigment-based ink, and combinations thereof.

12. The device of claim 1, wherein the device is provided in a configuration selected from the group consisting of a roll form, a strip form, and a set of pre-cut lengths.

13. The device of claim 12, wherein the roll form of the device is housed in a roll dispenser.

14. The device of claim 12, wherein the strip form and set of pre-cut lengths of the device are housed in a sterile, hermetically sealed package.

15. A method of marking a skin surface to delineate a radiation therapy portal area, the method comprising the steps of:
providing at least one skin marker;
removing a portion of a backing liner of a skin marker to expose an adhesive surface of the skin marker;
positioning the at least one skin marker on a skin surface of a patient, wherein the positioning of the at least one skin marker delineates a radiation therapy portal area; and
releasably securing the at least one skin marker in place on the skin surface of the patient.

16. The method of claim 15, wherein the radiation therapy portal area comprises areas selected from the group consisting of a perimeter, an isocenter and at least one set-up point.

17. The method of claim 15, wherein application of pressure to the at least one skin marker causes the adhesive surface of the skin marker to releasably adhere to the skin surface of the patient.

18. The method of claim 17, wherein the skin maker is retainable on the skin surface of a patient for a time period in the range from about seven to about ten days.
